(19) United States
(12) Patent Application Publication
(10) Pub. No.: US 2026/0339080 A1
(43) Pub. Date: Oct. 29, 2026

(54) CONTACT-TYPE MINIATURIZED OCULAR SURFACE IMAGING SYSTEM

(71) Applicants: POSTECH RESEARCH AND BUSINESS DEVELOPMENT FOUNDATION, Pohang (KR); CELLSHINE CO., LTD., Gwacheon-si (KR); SEOUL NATIONAL UNIVERSITY HOSPITAL, Seoul (KR)

(72) Inventors: Ki Hean KIM, Pohang (KR); Ji Eun YUN, Pohang (KR); Su Il JEON, Pohang (KR); No Seong PARK, Pohang (KR); Ji Sang LEE, Pohang (KR); Ki Yeon KIM, Gwacheon-si (KR); Ja Hueck KOO, Gwacheon-si (KR); Chang Ho YOON, Seoul (KR)

(73) Assignees: POSTECH RESEARCH AND BUSINESS DEVELOPMENT FOUNDATION, Pohang (KR); CELLSHINE CO., LTD., Gwacheon-si (KR); SEOUL NATIONAL UNIVERSITY HOSPITAL, Seoul (KR)

(21) Appl. No.: 19/658,555

(22) Filed: Apr. 25, 2026

(30) Foreign Application Priority Data
Apr. 25, 2025 (KR) 10-2025-0054497
Apr. 21, 2026 (KR) 10-2026-0071792

(51) Int. Cl. G02B 13/00; A61B 3/00; A61B 3/14; G02B 21/16; G02B 27/14; H04N 23/55; H04N 23/56

(52) U.S. Cl. CPC G02B 13/0075; A61B 3/0083; A61B 3/14; G02B 21/16; G02B 27/141; H04N 23/55; H04N 23/56

(57) ABSTRACT

Proposed is a contact-type miniaturized ocular surface imaging system, wherein the contact-type miniaturized ocular surface imaging system, includes an imaging module configured to illuminate an ocular surface with excitation light and acquire an image from excited emission light, and the imaging module includes a light source part configured to generate the excitation light; an image part configured to generate the image from the emission light; an optical transfer part through which the excitation light and the emission light travel between the light source part, the ocular surface, and the image part; and an end cap part provided between the optical transfer part and the ocular surface, and coming into direct contact with the ocular surface.

### CROSS REFERENCE TO RELATED APPLICATION

The present application claims priority to Korean Patent Application Nos. 10-2025-0054497 filed on Apr. 25, 2025 and 10-2026-0071792 filed on Apr. 21, 2026, the entire contents of which are incorporated herein for all purposes by these references.

### ACKNOWLEGEMENT

National Research and Development Project Supporting the Present Disclosure

Project Number: RS-2025-17492968

Ministry Name: Ministry of Science and ICT; Ministry of Education

Project Management (Specialized) Institution: National Research Foundation of Korea (NRF)

Research Topic: Oblique Light Sheet Microscope for Rapid Non-Invasive Examination of Human Conjunctival Goblet Cells

Project Performing Institution: Seoul National University Hospital(SNUH)

Research Period: From Jun. 01, 2023 to May 31, 2026

This work was additionally supported by Samsung Electronics (SRFC-IT2101-05).

### BACKGROUND OF THE INVENTION

### Field of the Invention

The present disclosure relates to a contact-type miniaturized ocular surface imaging system. More particularly, the present disclosure relates to a contact-type fluorescence microscope-based ocular surface imaging system capable of imaging goblet cells with high contrast and high resolution by using a small end cap that may come into direct contact with various regions of the ocular surface including the conjunctiva.

### Description of the Related Art

The ocular surface consists of a structure including the cornea and the conjunctiva, and serves an important role in protecting the eyeball from the external environment and maintaining visual function. In particular, goblet cells, which secrete mucus, are distributed in the conjunctiva epithelium and perform main functions in stabilizing the tear film and maintaining ocular surface homeostasis. Changes in the density and morphology of such goblet cells are known as major biomarkers for various ocular surface diseases, such as dry eye syndrome, inflammatory conjunctival diseases, and autoimmune diseases.

Accordingly, various inspection and imaging technologies for precisely observing and quantifying conjunctival goblet cells have been proposed. Typically, impression cytology has been used in clinical settings, which is a method of collecting cells by enabling a filter to come into contact with the conjunctival surface and then performing staining and microscopic observation. However, since such impression cytology is an invasive method of physically collecting cells, it may cause pain and discomfort during inspection. Furthermore, sample preparation and analysis processes are complicated and the inspection time is long, making it difficult to perform repeated measurements. In addition, since cells are collected only from a limited region, it is difficult to represent the overall condition of the conjunctiva.

In the meantime, in vivo confocal microscopy (IVCM) is a technology capable of imaging ocular tissues at high resolution in vivo, and may be utilized for observing the cornea and the conjunctiva. However, since IVCM is an imaging method based on reflection contrast, it has a low image contrast for goblet cells in structurally complex conjunctival tissues, making clear distinction difficult. Furthermore, since its field of view is significantly narrow, it is difficult to continuously image several conjunctival regions of the same subject. In addition, high equipment costs and complex operation impose constraints on its utilization as routine clinical diagnostic equipment.

Recently, fluorescence-based non-contact-type ocular surface imaging technologies have been proposed. In particular, research has been reported on selectively imaging conjunctiva goblet cells by using the intrinsic fluorescence characteristics of moxifloxacin, which is an antibiotic. Such non-contact-type fluorescence imaging system have the advantage of being able to capture a relatively wide area quickly. However, due to their structural characteristics as well as eyelid and anatomical constraints, imaging is mainly possible only for the inferior bulbar conjunctiva, and it is difficult to access the superior bulbar, fornix, nasal, and temporal conjunctival areas. Furthermore, in order to stably image the curved conjunctival surface in a non-contact state, high-speed scanning and surface-tracking mechanisms are required, which leads to complex system structure and increased costs.

As described above, conventional ocular surface inspection and imaging technologies commonly have problems such as invasiveness, low image contrast for goblet cells, limited imageable conjunctival areas, system complexity, and high costs. In particular, there has been a continuous demand for an imaging system having a simple structure that can stably image goblet cells distributed in various conjunctival regions with high contrast and high resolution, while also enabling repeated examinations and clinical application.

However, conventional technologies alone have not sufficiently provided technical means for non-invasively and stably imaging goblet cells across multiple areas of the conjunctiva.

The foregoing is intended merely to aid in the understanding of the background of the present disclosure, and is not intended to mean that the present disclosure falls within the purview of the related art that is already known to those skilled in the art.

### Document of Related Art

(Patent Document 1) Korean Patent No. 10-2146087 (Title of the Invention: DIGITAL MICROSCOPE OF CONTACT TYPE FOR OPHTHALMOLOGY, publication date: 19 Aug. 2020)

### SUMMARY OF THE INVENTION

The present disclosure is directed to providing a contact-type miniaturized ocular surface imaging system capable of stably imaging goblet cells distributed in various regions of the conjunctiva with high contrast and high resolution, while minimizing invasiveness, limitations in imaging areas, and system complexity.

The technical problems to be solved by the present disclosure are not limited to the aforementioned technical problems, and other technical problems which are not mentioned will be clearly understood by those skilled in the art from the following description

According to the present disclosure, there is provided a contact-type miniaturized ocular surface imaging system including an imaging module configured to illuminate an ocular surface with excitation light and acquire an image from excited emission light, wherein the imaging module includes: a light source part configured to generate the excitation light; an image part configured to generate the image from the emission light; an optical transfer part through which the excitation light and the emission light travel between the light source part, the ocular surface, and the image part; and an end cap part provided between the optical transfer part and the ocular surface, and coming into direct contact with the ocular surface.

The contact-type miniaturized ocular surface imaging system may further include a positioning module configured to move the imaging module in X, Y, and Z directions and rotate the imaging module along a particular arc.

In addition, the optical transfer part may include: an objective lens coupled to the end cap part; an excitation light transfer part configured to enable the excitation light to travel such that the excitation light reaches the objective lens; an emission light transfer part configured to enable the emission light to travel such that the emission light reaches the image part; and a dichroic mirror configured to selectively transmit or reflect the excitation light and the emission light.

In addition, the excitation light transfer part or the emission light transfer part may include a depth-of-focus adjustment part configured to adjust a depth of focus.

In addition, the depth-of-focus adjustment part may include an electronic tunable lens or a tube lens position adjustment part.

In addition, the excitation light transfer part or the emission light transfer part may include a depth extension adjustment part configured to extend a depth of focus.

In addition, the depth extension adjustment part may include a binary phase filter or a phase plate-based depth extension module.

In addition, the end cap part may include: an end cap body having a cylindrical shape or a truncated cone shape; an objective lens coupling part provided at a first side of the end cap body and coupled to an objective lens; and a transparent window provided at a second side of the end cap body and coming into contact with the ocular surface.

In addition, a transparent window may be made of a biocompatible material, may have an elasticity of a particular value or more, may be formed in a circular or polygonal form, may have a greater size than an imaging area of the image part, and may have a connection portion with respect to an end cap body, the connection portion being formed with a curved surface structure.

In addition, the positioning module may include: a 3D stage configured to move the imaging module in the X, Y, and Z directions; and a rotation stage configured to rotate the imaging module in the Z direction.

In addition, the rotation stage may include a rotation shaft and a rotation plate mounted on an end of the rotation shaft, and the imaging module may be disposed on an upper surface of the rotation plate at a position farther from a center of the ocular surface than the rotation shaft, along a straight line connecting the center of the ocular surface and the rotation shaft, and may be configured to rotate according to a curvature of the ocular surface.

In addition, the image part may include a camera of a smart device.

In addition, when the image part is the camera of the smart device, the contact-type miniaturized ocular surface imaging system may further include, between the optical transfer part and the image part, a smart device connection part configured to be connected to the camera of the smart device; or a smart device focus adjustment part configured to adjust a focus of the smart device.

In addition, a wavelength of the excitation light may be 405 nm, and the emission light may be a fluorescence signal caused by moxifloxacin.

In addition, the contact-type miniaturized ocular surface imaging system may further include an image processing part configured to compute density, size, and shape indexes of goblet cells of the ocular surface by applying a transformer-based cell segmentation algorithm to the image acquired by the image part.

The contact-type miniaturized ocular surface imaging system according to the present disclosure can achieve one or more of the following effects.

First, the contact-type miniaturized ocular surface imaging system according to the present disclosure stably comes into contact with the conjunctival surface using a contact-type end cap having a small transparent window, and thus can image the superior bulbar, inferior fornix, nasal, and temporal conjunctival areas, which are difficult to access with conventional non-contact-type imaging equipment, thereby conducting a comprehensive inspection of multiple areas of the conjunctiva.

Second, according to the present disclosure, by applying a fluorescence microscopic optical system that uses the intrinsic fluorescence characteristics of moxifloxacin, goblet cells can be selectively imaged with high contrast relative to conjunctival epithelial cells, and this can fundamentally address low cell identifiability that was a drawback in conventional technologies based on reflection contrast.

Third, the present disclosure is configured such that the focal plane is automatically aligned near the conjunctival epithelium through only slight contact with the end cap, enabling stable focus maintenance even without complex surface tracking or rapid scanning. This can simplify the system structure and reduce equipment costs and operational complexity.

Fourth, according to the present disclosure, a high-resolution optical system and real-time image acquisition at tens of frames per second can be secured, thus providing robust imaging against fine movement of the eyeball. This enables repetitive and highly reliable inspections in a clinical environment.

Fifth, according to the present disclosure, quantitative analysis of the density, size, and morphology of goblet cells can be automatically conducted on the basis of an acquired fluorescence image, thus enabling the diagnosis and treatment progress for dry eye syndrome, inflammatory conjunctival diseases, and autoimmune diseases to be evaluated using objective numerical values.

Sixth, according to the present disclosure, repetitive imaging in the same subject or animal model is enabled through a non-invasive contact method, thus enabling precise tracking of goblet cell changes over time during preclinical research and a drug efficacy evaluation process.

Effects that may be obtained from the present disclosure will not be limited to only the above described effects. In addition, other effects which are not described herein will become apparent to those skilled in the art from the following description.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a diagram illustrating a configuration of an imaging module of a contact-type miniaturized ocular surface imaging system according to the present disclosure;

FIG. 2 is a diagram illustrating a configuration of a positioning module of a contact-type miniaturized ocular surface imaging system according to the present disclosure;

FIG. 3 is a diagram illustrating a configuration of an optical transfer part of a contact-type miniaturized ocular surface imaging system according to the present disclosure;

FIG. 4 is a diagram illustrating a configuration of an end cap part of a contact-type miniaturized ocular surface imaging system according to the present disclosure;

FIG. 5 is a diagram illustrating a configuration of a smart device of a contact-type miniaturized ocular surface imaging system according to the present disclosure;

FIG. 6 is a diagram illustrating an overall configuration of a contact-type miniaturized ocular surface imaging system according to the present disclosure;

FIG. 7 shows diagrams (A)-(B) illustrating a transformer-based segmentation method of a contact-type miniaturized ocular surface imaging system according to the present disclosure; and

FIG. 8 is a diagram illustrating changes in optical performance according to the window thickness of an end cap part of a contact-type miniaturized ocular surface imaging system according to the present disclosure.

### DETAILED DESCRIPTION OF THE INVENTION

Advantages and features of the present disclosure, and methods to achieve them will be apparent from the following embodiments that will be described in detail with reference to the accompanying drawings. It should be understood that the present disclosure is not limited to the following embodiments and may be embodied in different ways, and that the embodiments are provided only to make the disclosure of the present disclosure complete and to provide a thorough understanding of the present disclosure to those skilled in the art. The present disclosure is defined only by the scope of the claims. Throughout the description, the same reference numerals refer to same elements.

The size or shape of the elements shown in the drawings attached to the present specification may be exaggerated for clarity and convenience of description. It should be noted that the same reference numerals will be used throughout the drawings to refer to the same elements. In addition, detailed descriptions of functions and configurations of well-known technologies that are determined to unnecessarily obscure the gist of the present disclosure may be omitted.

Terms used in the present specification is for the purpose of describing particular embodiments only and is not intended to be limiting of the present disclosure. As used in the present specification, the singular forms are intended to include the plural forms as well, unless the context clearly indicates otherwise. In addition, throughout the present specification, when a part “includes” an element, this means that the part may further include other elements, unless specifically stated otherwise.

It will be understood that when an element is referred to as being “coupled” or “connected” to another element, it can be directly coupled or connected to the other element or intervening elements may be present therebetween. In contrast, it will be understood that when an element is referred to as being “directly coupled” or “directly connected” to another element, there are no intervening elements present. Other expressions for describing the relationship between elements should be interpreted in the same manner.

Terms such as top, bottom, upper surface, lower surface, upper portion, or lower portion used in the present specification are used to distinguish relative positions of elements. For example, when an upper side of a drawing is referred to as an upper portion and a lower side of the drawing is referred to as a lower portion for convenience, the upper portion may be referred to as a lower portion and the lower portion may be referred to as an upper portion in practice, without departing from the scope of the present disclosure.

Terms including ordinal numbers such as first, second, and the like used in the present specification may be used to describe various elements, but do not limit the elements. These terms are only used to distinguish each element from other elements and are not limited by an order of manufacture, and the names thereof in the detailed description and the claims may not be consistent with each other.

Unless otherwise defined, all terms including technical and scientific terms used in the present specification have the same meanings as commonly understood by those skilled in the art. It will be further understood that terms used herein should be interpreted as having a meaning that is consistent with their meaning in the context of this specification and the relevant art and will not be interpreted in an idealized or overly formal sense unless expressly so defined herein.

Hereinafter, a contact-type miniaturized ocular surface imaging system according to an embodiment of the present disclosure will be described with reference to the drawings.

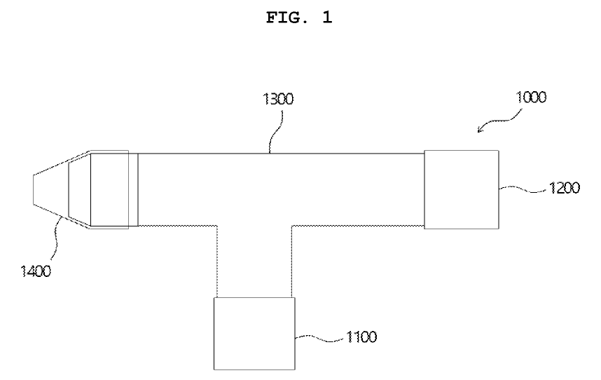

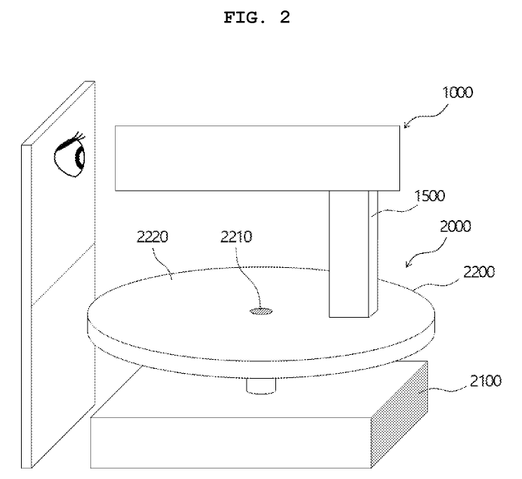

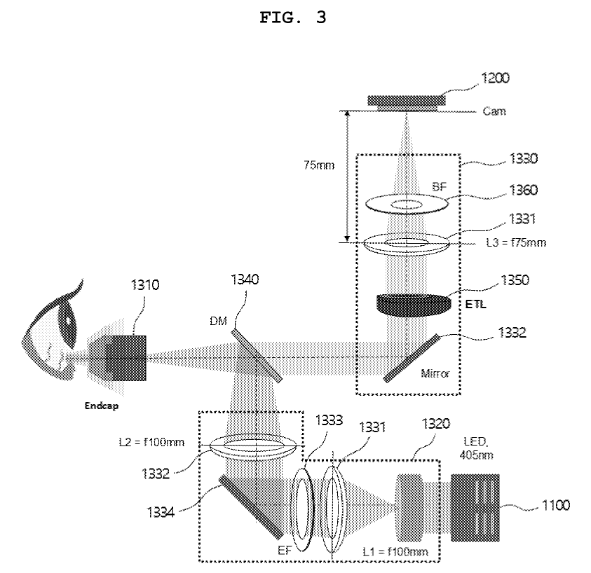

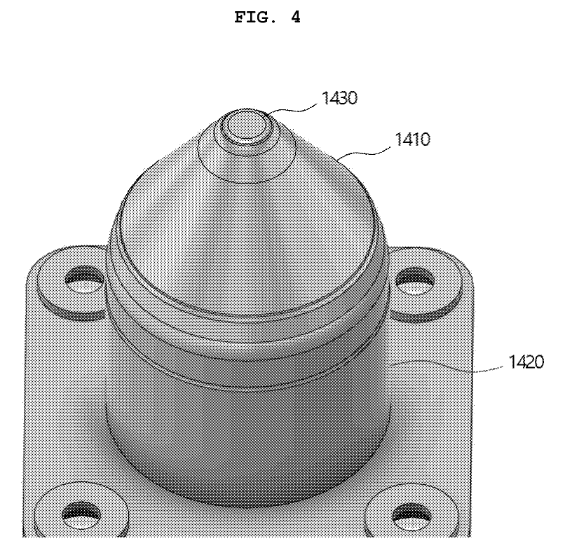

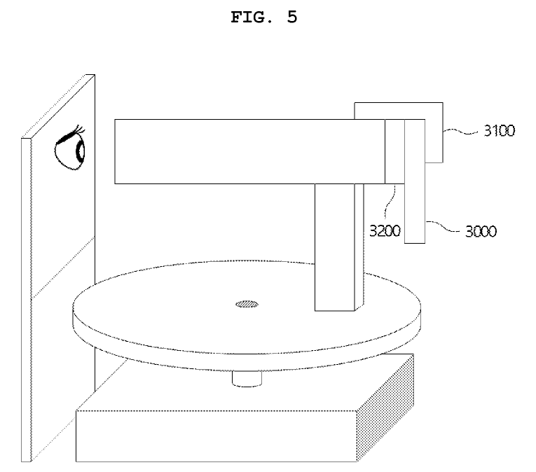

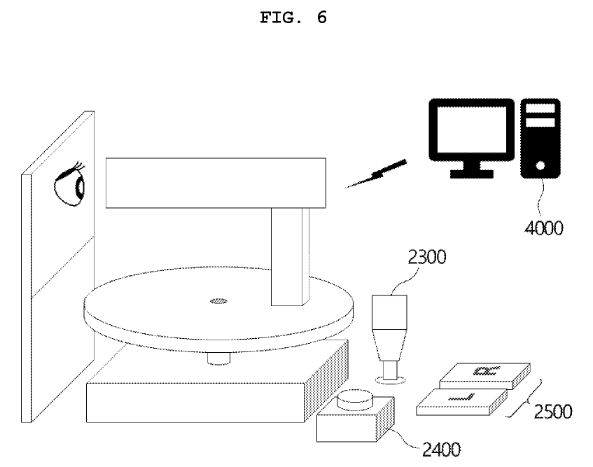

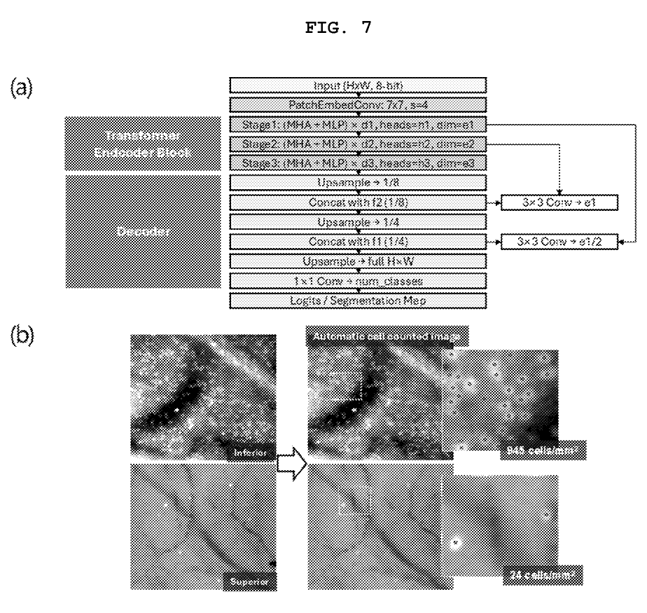

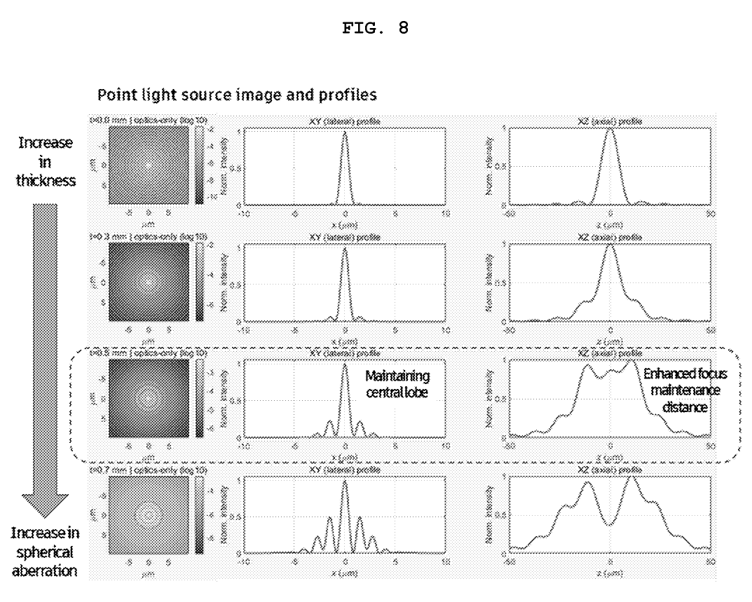

FIG. 1 is a diagram illustrating a configuration of an imaging module 1000 of a contact-type miniaturized ocular surface imaging system according to the present disclosure, FIG. 2 is a diagram illustrating a configuration of a positioning module 2000 of a contact-type miniaturized ocular surface imaging system according to the present disclosure, FIG. 3 is a diagram illustrating a configuration of an optical transfer part 1300 of a contact-type miniaturized ocular surface imaging system according to the present disclosure, and FIG. 4 is a diagram illustrating a configuration of an end cap part 1400 of a contact-type miniaturized ocular surface imaging system according to the present disclosure. In addition, FIG. 5 is a diagram illustrating a configuration of a smart device 3000 of a contact-type miniaturized ocular surface imaging system according to the present disclosure, FIG. 6 is a diagram illustrating an overall configuration of a contact-type miniaturized ocular surface imaging system according to the present disclosure, FIGS. 7A and 7B are diagrams illustrating a transformer-based segmentation method of a contact-type miniaturized ocular surface imaging system according to the present disclosure, and FIG. 8 is a diagram illustrating changes in optical performance according to the window thickness of an end cap part of a contact-type miniaturized ocular surface imaging system according to the present disclosure.

Referring to FIG. 1, a contact-type miniaturized ocular surface imaging system according to the present disclosure includes an imaging module 1000 for acquiring an image from emission light excited by illuminating an ocular surface with excitation light. The imaging module 1000 includes: a light source part 1100 for generating excitation light; an image part 1200 for generating an image from emission light generated from the ocular surface; an optical transfer part 1300 that is an optical path part including a plurality of lenses and reflectors to enable the excitation light and the emission light to travel between the light source part 1100, the ocular surface, and the image part 1200; and an end cap part 1400 provided between the optical transfer part 1300 and the ocular surface, and coming into direct contact with the ocular surface.

The light source part 1100 may include a light-emitting diode, a laser diode, or an equivalent light source thereof, and is configured to generate excitation light to illuminate the ocular surface with the excitation light and transfer the excitation light to the ocular surface through the optical transfer part 1300.

The image part 1200 is an element for generating an image by receiving emission light generated from the ocular surface, and may include a CMOS image sensor, a CCD image sensor, or an image sensor functionally equivalent thereto.

The optical transfer part 1300 is an optical path part for guiding the excitation light generated from the light source part 1100 to reach the ocular surface through the end cap part 1400 while simultaneously guiding the emission light generated from the ocular surface to reach the image part 1200. The optical path part includes a plurality of lenses, reflectors, filters, and dichroic mirrors, and controls travel paths of the excitation light and the emission light.

The end cap part 1400 is an element coupled to an end of the optical transfer part 1300 to come into direct contact with the ocular surface, and includes a transparent window to enable the ocular surface to be illuminated with the excitation light and enable the emission light to return to the optical transfer part 1300, and is configured to stably maintain an image focal plane through contact with the ocular surface.

Accordingly, the contact-type miniaturized ocular surface imaging system according to the present disclosure may acquire a high-resolution image in a stable contact state with the ocular surface, and enables reliable imaging even without a complex non-contact-type surface tracking structure.

Referring to FIG. 2, the contact-type miniaturized ocular surface imaging system according to the present disclosure may further include a positioning module 2000 for moving the imaging module 1000 in X-axis, Y-axis, and Z-axis directions and rotating the same along in a particular arc.

The positioning module 2000 is an element for precisely adjusting the position and the orientation of the imaging module 1000 to correspond to various regions of the ocular surface, and is configured to finely move the imaging module 1000 in an X-axis which is a left-right direction, a Y-axis which is a forward-backward direction, and a Z-axis which is an upward-downward direction.

In addition, the positioning module 2000 is configured to rotate the imaging module 1000 at a particular angle according to the curvature of the ocular surface such that the end cap part 1400 may maintain a state of being in close contact with the ocular surface.

Accordingly, through the positioning module 2000, a user may adjust the position and the angle of the imaging module 1000 without changing a posture of a subject, so as to stably access various regions of the ocular surface, such as the inferior bulbar conjunctiva, the superior bulbar conjunctiva, fornix, nasal, and temporal conjunctival areas.

According to the present disclosure, the positioning module 2000 can precisely control the position and adjust the angle of the imaging module 1000, thereby improving stability and reproducibility in the contact-type imaging process.

Referring to FIG. 3, in the contact-type miniaturized ocular surface imaging system according to the present disclosure, the optical transfer part 1300 includes: an objective lens 1310 coupled to the end cap part 1400; an excitation light transfer part 1320 for enabling the excitation light to travel such that the excitation light reaches the objective lens 1310; an emission light transfer part 1330 for enabling the emission light to travel such that the emission light generated from the ocular surface reaches the image part 1200; and a dichroic mirror 1340 for selectively transmitting or reflecting the excitation light and the emission light.

The objective lens 1310 is an optical element for observing the ocular surface through the transparent window of the end cap part 1400, and is configured to focus the excitation light with which the ocular surface is illuminated and collect the emission light generated from the ocular surface to transfer the same into the optical transfer part 1300.

The excitation light transfer part 1320 is an optical path part for guiding the excitation light generated from the light source part 1100 to reach the objective lens 1310, and includes a plurality of lenses, reflectors, and optical filters, and is configured to control a traveling direction and an optical axis of the excitation light.

The emission light transfer part 1330 is an optical path part for guiding the emission light generated from the ocular surface to reach the image part 1200 after being collected through the objective lens 1310, and includes a plurality of lenses, reflectors, and fluorescence filters, and is configured to enable wavelength selection and efficient transfer of the emission light.

The dichroic mirror 1340 is an optical element for selectively transmitting or reflecting light according to wavelength characteristics of the excitation light and the emission light, and is disposed to reflect the excitation light toward the objective lens 1310 and transmit the emission light toward the image part 1200, or vice versa.

Accordingly, the optical transfer part 1300 is configured to enable the excitation light and the emission light to travel along separated paths without mutual interference while sharing the same optical axis, thereby enabling stable light illumination and high-efficiency emission light collection in the contact-type ocular surface imaging process.

Referring back to FIG. 3, in the contact-type miniaturized ocular surface imaging system according to the present disclosure, the excitation light transfer part 1320 or the emission light transfer part 1330 may include a depth-of-focus adjustment part 1350 capable of adjusting a depth of focus.

The depth-of-focus adjustment part 1350 is an element for preventing a portion of an image from being in a defocused state due to a curvature change and a fine up-down movement of the ocular surface, and is disposed on an optical path of the optical transfer part 1300, and is configured to actively adjust an effective focus position of an optical system.

In an embodiment of the present disclosure, the imaging module 1000 includes the objective lens 1310 having a numerical aperture (NA) of about 0.3 or more. When such a high-NA objective lens is used, an optical depth of focus is formed very short, at about 20 μm or less.

Accordingly, in an object having a curved surface structure such as the ocular surface and in which a fine movement occurs during inspection, a problem may occur in which a partial area easily falls into an out-of-focus state. Therefore, the depth-of-focus adjustment part 1350 operates to compensate for such defocus in real time or near real time.

Specifically, the depth-of-focus adjustment part 1350 is configured to finely move an optical image-forming position on an optical path such that a focal plane of the objective lens 1310 is maintained near an epithelium layer of the ocular surface, thereby securing uniform focus quality over an entire area of an image even in the contact-type imaging process.

According to the present disclosure, a high-resolution optical system is maintained and robust focus stability is provided against a fine curvature and movement of the ocular surface, thereby improving reliability of precise imaging and quantitative analysis of goblet cells.

In the contact-type miniaturized ocular surface imaging system according to the present disclosure, the depth-of-focus adjustment part 1350 may include an electronic tunable lens or a tube lens position adjustment part.

The electronic tunable lens is an optical element capable of continuously changing the refraction of the lens according to an applied electrical signal, and is configured to rapidly adjust an effective focus position of an optical system without mechanical movement.

In an embodiment of the present disclosure, the electronic tunable lens is capable of changing a focus position with a response speed in units of several milliseconds (ms), and is configured to enable focus tracking even in a real-time imaging environment with an image acquisition speed of about 14 frames/second (fps) or more.

In another embodiment, the depth-of-focus adjustment part 1350 may include a tube lens position adjustment part for finely moving, in an optical axis direction, the position of a tube lens disposed near the image part 1200, and the tube lens position adjustment part may be configured to have precision of movement in units of micrometers.

In this case, when the objective lens 1310 has a magnification of about 10 times (10×) and the focal distance of a relay lens is about 75 mm and the image part 1200 is a CMOS sensor having a pixel size of about 1.4 μm, a focus position may be precisely adjusted through the tube lens position adjustment part while maintaining a spatial resolution at a level of about 1.0 μm over an entire imaging area (field of view) in a range of about 1.8 mm × 1.3 mm.

Accordingly, the depth-of-focus adjustment part 1350 including the electronic tunable lens or the tube lens position adjustment part may provide stable images with high resolution and high contrast without separate mechanical scanning in the contact-type ocular surface imaging process, and reduces an operational burden on an examiner and simplifies the structure of the entire system.

In the contact-type miniaturized ocular surface imaging system according to the present disclosure, the excitation light transfer part 1320 or the emission light transfer part 1330 may include a depth extension adjustment part 1360 for extending the depth of focus.

The depth extension adjustment part 1360 is an element for making up for a problem in which the depth of focus is limited to about 20 μm or less due to a high numerical aperture (NA 0.3 or more) of the objective lens 1310, and is configured to artificially extend an optical depth of focus such that a focus is maintained over an entire imaging area in an object having a curvature such as the ocular surface.

Specifically, the depth extension adjustment part 1360 may be designed to be disposed on an optical path of the optical transfer part 1300 to transform a form of a point spread function, thereby enabling an extension of a depth of focus by about 2 to 3 times or more compared to a conventional one. Accordingly, an effective depth of focus at a level of about 40 μm to 60 μm may be secured.

By the application of the depth extension adjustment part 1360, goblet cells may be observed in a simultaneously in-focus state over an entire imaging area despite a fine curved surface structure of the conjunctival surface or a height deviation occurring in a contact state.

According to the present disclosure, a local out-of-focusing problem that may frequently occur in the contact-type imaging process may be minimized through the depth extension adjustment part 1360, and a high-definition image of a large area may be acquired without separate mechanical focus scanning.

In the contact-type miniaturized ocular surface imaging system according to the present disclosure, the depth extension adjustment part 1360 may include a binary phase filter or a phase plate-based depth extension module.

The binary phase filter is a phase element designed to modulate a phase of incident light on an optical path into discrete values such as 0 or π, and is configured to increase a depth of focus by transforming a point spread function of light passing through the objective lens 1310 into an intentionally expanded form.

Alternatively, the phase plate-based depth extension module operates by reducing focus sensitivity in an optical axis direction using a phase plate having a continuous phase distribution. In this case as well, it may be designed to enable an extension of a depth of focus by about 3 times compared to a conventional one.

The binary phase filter or the phase plate-based depth extension module may be inserted on an optical path of the excitation light transfer part 1320 or the emission light transfer part 1330, and may be configured to compensate for resolution degradation due to the extended point spread function by applying a deconvolution algorithm after image acquisition.

According to the present disclosure, by combining optical depth extension and computational image reconstruction techniques, uniform focus quality over an entire conjunctival area and a spatial resolution at a level of about 1.0 μm may be simultaneously achieved while maintaining a contact-type miniaturized structure.

Referring to FIG. 4, in the contact-type miniaturized ocular surface imaging system according to the present disclosure, the end cap part 1400 may include: an end cap body 1410 having a cylindrical shape or a truncated cone shape; an objective lens coupling part 1420 formed at a first side of the end cap body 1410 and coupled to the objective lens 1310; and a transparent window 1430 formed at a second side of the end cap body 1410 and configured to come into direct contact with the ocular surface.

The end cap body 1410 may be formed of a transparent or translucent material applicable to medical devices, preferably polymethyl methacrylate (PMMA), and its overall outer shape is designed in a cylindrical shape or a truncated cone shape to be smoothly inserted and accessible to the anatomical structure of the eyeball and the eyelid. The truncated cone shape may facilitate smooth access to a narrow conjunctival area.

The objective lens coupling part 1420 is formed at the first side of the end cap body 1410, and is configured to be coupled to the objective lens 1310 without an alignment error by being provided with at least one selected from the group of a screw-type, a sliding-type, and a press-fit type coupling structure.

The transparent window 1430 may be formed at the second side of the end cap body 1410 as a single object or coupled as a separate member, and as a part that comes into direct contact with the ocular surface, the transparent window is formed with a structure having high transmittance so that excitation light and emission light may be transmitted without loss.

An inner diameter of the transparent window 1430 is formed to be about 3.4 mm or more to prevent optical vignetting during imaging, and an outer diameter of the transparent window is formed to be about 4.5 mm to minimize mechanical interference by the eyelid. The total thickness is formed in a range of about 0.5 mm to 0.7 mm to simultaneously satisfy miniaturization and weight reduction. In addition, by having a minimum thickness, induction of optical aberration may also be minimized.

Referring to FIG. 8, it can be found that optical performance (optical aberration) changes according to the thickness of the transparent window 1430, and it can be seen that as the thickness increases, spherical aberration increases and resolution degrades.

In addition, it can be seen that the FWHM of the central lobe is maintained without significant change over the entire range of about 0 to 0.7 mm, and at about 0.5 mm or less, the dominant structure of the PSF maintains a core-lobe (core-dominated) form, whereas in the range of 0.6 to 0.7 mm, the side-lobe amplitude increases. Considering the aberration correction design of the objective lens caused by a 0.17 mm-thick coverslip, it can be seen that robustness against optical aberration is maintained up to a window thickness of 0.7 mm.

Therefore, considering optical performance and minimum manufacturability (if it is too thin, processing is impossible and durability decreases due to easy tearing), a thickness of the transparent window 1430 is preferably 0.7 mm.

Accordingly, in the present disclosure, the end cap part 1400 serves as a physical interface between an imaging optical system and the ocular surface, and enables acquisition of a high-resolution image without separate complex focus adjustment by enabling an image focal plane to be automatically positioned near the ocular surface in a stable contact state.

In the contact-type miniaturized ocular surface imaging system according to the present disclosure, the transparent window 1430 is made of a biocompatible material, and has an elasticity of a particular value or more, and is formed in a circular or polygonal form, and has a greater size than an imaging area of the image part 1200, and has a connection portion with respect to the end cap body 1410, wherein the connection portion may be formed with a curved surface structure.

The transparent window 1430 is an element that comes into direct contact with the ocular surface, so it is formed of a material with verified biocompatibility, and preferably, medical-grade PMMA, silicone-coated glass, or an equivalent elastic transparent material may be used.

The transparent window 1430 is designed to have an elasticity of a particular level or more to prevent concentration of local pressure when being in contact with the ocular surface, and is configured to minimize damage to the conjunctival epithelium and a subject's foreign body sensation.

The transparent window 1430 may be formed in a circular or polygonal shape, and its diameter or outer dimension is set to be larger than that of an effective imaging area of the image part 1200, thereby preventing optical shielding and distortion that may occur at an edge of an image.

In addition, a connection part between the transparent window 1430 and the end cap body 1410 is formed with a curved surface structure to prevent a foreign body sensation or conjunctival irritation caused by sharp edges when being in contact with the ocular surface, and enables contact pressure to be distributed over a wide area.

Accordingly, in the present disclosure, the transparent window 1430 secures ocular safety and user convenience in the contact-type imaging process, while simultaneously improving image quality by minimizing optical loss of excitation light and emission light.

Referring back to FIG. 2, in the contact-type miniaturized ocular surface imaging system according to the present disclosure, the positioning module 2000 may include: a 3D stage 2100 for moving the imaging module 1000 in X-axis, Y-axis, and Z-axis directions; and a rotation stage 2200 for rotating the imaging module 1000 around the Z-axis direction.

The 3D stage 2100 is configured to finely adjust the position of the imaging module 1000 in the X-axis which is a left-right direction, the Y-axis which is a forward-backward direction, and the Z-axis which is an upward-downward direction, independently, thereby enabling precise access to various positions on the ocular surface.

The rotation stage 2200 is configured to perform rotation operation around the Z-axis such that the imaging module 1000 is tilted according to the curvature of the ocular surface. Accordingly, the end cap part 1400 maintains a constant contact state with respect to the ocular surface.

Accordingly, the positioning module 2000 may precisely control the position and the orientation of the imaging module 1000 in the contact-type imaging process by combining linear movement by the 3D stage 2100 and rotational movement by the rotation stage 2200.

Referring back to FIG. 2, in the contact-type miniaturized ocular surface imaging system according to the present disclosure, the rotation stage 2200 may include a rotation shaft 2210, and a rotation plate 2220 mounted on an end of the rotation shaft 2210.

The rotation shaft 2210 is supported by the positioning module 2000, and the rotation plate 2220 is configured to be rotatable around the rotation shaft 2210.

A support member 1500 of the imaging module 1000 is disposed on an upper surface of the rotation plate 2220 at a position farther than the rotation shaft 2210, along a straight line connecting the rotation shaft 2210 and the center of the ocular surface.

By disposing the support member 1500 of the imaging module 1000 as a position spaced apart from the center of the rotation shaft 2210 in this manner, the imaging module 1000 may be configured to move along an arc path corresponding to the curvature of the ocular surface when the rotation stage 2200 rotates.

Accordingly, the end cap part 1400 may approach the ocular surface in a tangential direction or maintain a close contact state even during the rotation operation, thereby preventing excessive pressure concentration or slippage that may occur in the contact-type imaging process.

According to the present disclosure, a structural arrangement of the rotation stage 2200 enables a natural rotational movement along the curvature of the ocular surface, thereby enabling stable and highly reproducible contact imaging even in a region that is difficult to access, such as a superior bulbar conjunctiva or a fornix area.

Referring to FIG. 5, in the contact-type miniaturized ocular surface imaging system according to the present disclosure, the image part 1200 may include a camera provided in a smart device (e.g., a smartphone or a tablet computer).

When the image part 1200 is a camera of a smart device, the image part 1200 includes a high-resolution CMOS image sensor, an auto-exposure control function, and a real-time image streaming function, and is configured to convert emission light generated from the ocular surface into image data.

Such a smart device-based image part 1200 is easier to miniaturize, has higher cost-effectiveness, and possesses superior portability compared to conventional dedicated industrial cameras, thus improving miniaturization and mobility of the overall equipment in a clinical or research environment.

In addition, image acquisition, real-time display, data storage, and post-processing functions may be performed in a single device by utilizing computational processing capability and storage function of the smart device, thereby enabling an integrated configuration of the imaging system without a separate external image processing device.

Referring back to FIG. 5, in the contact-type miniaturized ocular surface imaging system according to the present disclosure, when the image part 1200 is a camera of the smart device, a smart device connection part 3100 connected to the camera of the smart device or a smart device focus adjustment part 3200 for adjusting a focus of the smart device may be further included between the optical transfer part 1300 and the image part 1200.

The smart device connection part 3100 includes an adapter structure mechanically and optically coupled such that the exit optical axis of the optical transfer part 1300 and the optical axis of the camera lens of the smart device are aligned, and is configured to be detachable to be compatible with various models of smart devices.

The smart device focus adjustment part 3200 may be configured to finely adjust the position of the camera lens of the smart device or a focus position in conjunction with an internal focusing module, thereby compensating for a focus error that may occur due to a change in a contact state with the ocular surface or adjustment of an optical system.

According to the present disclosure, stable optical coupling and precise focus control are simultaneously secured, while applying the smart device-based image part 1200, thereby acquiring a highly reliable image of the ocular surface even in a clinical environment.

In the meantime, in the contact-type miniaturized ocular surface imaging system according to the present disclosure, the wavelength of the excitation light is 405 nm, and the emission light may be a fluorescence signal generated by moxifloxacin.

The excitation light is light in a 405 nm wavelength band, and is selected to correspond to a wavelength area in which moxifloxacin molecules have an absorption spectrum, and is stably generated by a light-emitting diode (LED) or a laser diode included in the light source part 1100.

Moxifloxacin is a fluoroquinolone antibiotic widely used as eye drops in the ophthalmic field, and has a characteristic of selectively accumulating in the ocular surface, particularly goblet cells the conjunctival epithelium. When moxifloxacin molecules are excited by 405 nm excitation light, the moxifloxacin molecules emit a fluorescence signal in a wavelength range of approximately 460 nm to 520 nm, which exhibits a relatively higher fluorescence intensity relative to surrounding conjunctival epithelial cells.

Accordingly, moxifloxacin accumulated in goblet cells is selectively excited by 405 nm excitation light with which the ocular surface is illuminated, to generate emission light, and the emission light is transferred to the image part(1200) through the optical transfer part 1300 and is converted into an image signal.

A dichroic mirror and a fluorescence emission filter are disposed in the optical transfer part 1300 considering wavelength characteristics of the excitation light and the emission light. Accordingly, 405 nm excitation light is efficiently reflected toward the ocular surface, and a fluorescence signal generated by moxifloxacin is selectively transmitted toward the image part 1200.

Through such a wavelength-selective optical configuration, according to the present disclosure, unlike a reflected light-based imaging method, only goblet cells may be selectively imaged using a high-contrast fluorescence signal while minimizing interference caused by structural complexity of tissue or surface reflection.

In particular, the imaging method using the 405 nm excitation light and the moxifloxacin fluorescence signal may secure safety and convenience of an inspection process simultaneously as clinically approved eye drops are used as they are without additional administration of a separate fluorescence dye.

According to the present disclosure, conjunctival goblet cells may be imaged with high contrast and a high signal-to-noise ratio through a combination of excitation light at a wavelength of 405 nm and a moxifloxacin-derived fluorescence signal. This provides a technical effect of significantly improving the accuracy of cell segmentation and quantitative analysis performed later by an image processing part.

Referring to FIGS. 7A and 7B, in the contact-type miniaturized ocular surface imaging system according to the present disclosure, the image processing part (not shown) is configured to receive a fluorescence image of the ocular surface acquired by the image part 1200 and apply a transformer-based cell segmentation algorithm, thereby computing density, size, and shape indexes of goblet cells distributed on the ocular surface.

The image processing part (not shown) may be implemented within a server 4000, and includes a transformer-based segmentation network. In order to automatically divide a cell area from an input image, the transformer-based segmentation network includes: an encoder that decomposes an input image into patches and extracts features; a decoder that restores the cell area by combining global and local features extracted by the encoder; and an output layer that finally classifies a goblet cell area on a pixel-by-pixel basis.

The encoder is configured to divide an input fluorescence image into patches of a particular size, and convert each patch into an embedding vector, and simultaneously learn global contextual information for the entire image area and local fluorescence features of goblet cells through a multi-head self-attention operation.

Herein, the encoder is configured to maintain spatial positional relationships between patches by including position embedding, and through this, a characteristic in which goblet cells are non-uniformly distributed throughout the conjunctival epithelium may be effectively reflected.

The decoder is configured to receive multi-scale feature maps output from the encoder and perform upsampling and feature combination processes, thereby generating a segmentation map having the same resolution as an input image.

The decoder includes an attention-based feature selection mechanism to clearly distinguish between a boundary area and an internal area of goblet cells while restoring, in a stepwise manner, features transferred from the encoder.

The image processing part is configured to calculate the density of goblet cells by computing the number of goblet cells per unit area on the basis of a pixel area classified as goblet cells from a segmentation result, and analyze the area, diameter, and aspect ratio of an individual goblet cell area to compute size and shape indexes.

In addition, the image processing part may be configured to remove noise caused by fine movement of the eyeball and provide a more stable quantitative result of goblet cells by performing temporal averaging or accumulative analysis on images acquired from multiple frames.

Accordingly, the image processing part according to the present disclosure does not rely on a user's subjective judgment and may automatically perform high-precision division and quantitative analysis of conjunctival goblet cells by utilizing transformer-based global context learning capability, thereby providing diagnosis and evaluation of treatment progress for dry eye syndrome, inflammatory eye diseases, and autoimmune diseases as objective indices.

Hereinafter, a detailed configuration of a contact-type miniaturized ocular surface imaging system according to an embodiment of the present disclosure will be described. This embodiment is for illustrative purposes only, and the scope of the present disclosure is not limited thereto.

Referring back to FIG. 3, the light source part 1100 may include a 405 nm LED, and excitation light generated from the light source part 1100 is transferred to the dichroic mirror 1340 via the excitation light transfer part 1320, and the transferred excitation light passes through the objective lens 1310 to illuminate the ocular surface. The above-described moxifloxacin is applied onto the ocular surface to express fluorescence, and the emission light excited by the 405 nm excitation light passes back through the dichroic mirror 1340 and passes through the emission light transfer part 1330 and is transferred to the image part 1200. Herein, an example of the image part 1200 is a camera.

In the meantime, the depth-of-focus adjustment part 1350 and the depth extension adjustment part 1360 may be provided in the excitation light transfer part 1320 or the emission light transfer part 1330, but are preferably provided in the emission light transfer part 1330. Herein, the excitation light transfer part 1320 and the light source part 1100 constitute an excitation stage, and the emission light transfer part 1330 and the image part 1200 constitute an image stage. Herein, emission light refers to emitted light, which is light emitted after fluorescence expression.

As an embodiment, in the emission light transfer part 1330, the emission light that has passed through the dichroic mirror 1340 passes through a mirror 1332, the depth-of-focus adjustment part 1350, an L3 lens (f 75 mm), and the depth extension adjustment part 1360, and is then transferred to the image part 1200. Herein, the depth-of-focus adjustment part 1350 may be an ETL, or a moving means capable of physically moving a focus position. In addition, the depth extension adjustment part 1360 may be a binary phase filter or a general phase filter. In the case of a binary phase filter or a general phase filter (tetrapod), the server 4000, which will be described later, may extend the depth of focus through image processing. As described above, the depth of focus may be extended by two to three times or more, thereby securing resolution up to a particular depth of the ocular surface.

In the excitation light transfer part 1320, the excitation light generated by the light source part 1100 passes through a L1 lens (f 100 mm), an EF 1333, a mirror 1334, a L2 lens (f 100 mm), the dichroic mirror 1340, and the objective lens 1310 to illuminate the ocular surface.

In the meantime, the above-described embodiment may be a straight-line structure without the mirrors 1332 and 1334 as an embodiment. Both the depth-of-focus adjustment part 1350 and the focus extension adjustment part 1360 may be provided, either one of them may be provided, or neither of them may be provided.

Referring back to FIG. 5, a case in which a smart device is used instead of the image part 1200 will be described as follows. The smart device 3000 may be a general smartphone or a tablet PC, and a camera provided in the smartphone or the tablet PC may be used. In this case, the smart device connection part 3100 for mounting or fixing the smart device may be provided, and the smart device connection part 3100 may be in a clamp type.

In the meantime, the smart device focus adjustment part 3200 may be a lens or a filter hardware-wise, or may be a focus adjustment function part for adjusting a focus within the smart device software-wise.

Referring back to FIG. 6, the positioning module 2000 according to the present disclosure may include a joystick 2300 for operating the 3D stage 2100, or may include a foot pedal 2500 for operating the rotation stage 2200. The joystick 2300 may move the stage in three dimensions. For example, movement in the X and Y directions may be adjusted by the direction of the joystick, and movement in the Z direction may be adjusted by the rotation direction of the joystick. The foot pedal 2500 may include a right pedal and a left pedal, and may be set such that the rotation stage may be rotated to the left (+z direction) when the right pedal is pressed, or the rotation stage may be rotated to the right (-z direction) when the left pedal is pressed. By adjusting the above-described foot pedal 2500, the imaging module 1000 may be rotated to follow the arc of the ocular surface.

Referring back to FIGS. 7A and 7B, the density of GCs may be calculated by transformer-based segmentation, and FIG. 7A shows a flowchart and FIG. 7B shows the results thereof. In this embodiment, it can be seen that the processing time for a 0.5 mm by 0.5 mm cropped image was 10 seconds, and the accuracy was measured at 93 %, indicating that the number and density were measured relatively accurately.

Although exemplary embodiments of the present disclosure have been illustrated and described above with reference to the drawings, the present disclosure is not limited to the above-described particular embodiments. It will be understood by those skilled in the art that various modifications may be made without departing from the gist of the present disclosure as claimed in the claims, and such modifications should not be individually understood from the technical idea or perspective of the present disclosure.

What is claimed is:

1. A contact-type miniaturized ocular surface imaging system, comprising:
an imaging module configured to illuminate an ocular surface with excitation light and acquire an image from excited emission light,
wherein the imaging module comprises:
a light source part configured to generate the excitation light;
an image part configured to generate the image from the emission light;
an optical transfer part through which the excitation light and the emission light travel between the light source part, the ocular surface, and the image part; and
an end cap part provided between the optical transfer part and the ocular surface, and coming into direct contact with the ocular surface.

2. The contact-type miniaturized ocular surface imaging system of claim 1, further comprising:
a positioning module configured to move the imaging module in X, Y, and Z directions and rotate the imaging module along a particular arc.

3. The contact-type miniaturized ocular surface imaging system of claim 1, wherein the optical transfer part comprises:
an objective lens coupled to the end cap part;
an excitation light transfer part configured to enable the excitation light to travel such that the excitation light reaches the objective lens;
an emission light transfer part configured to enable the emission light to travel such that the emission light reaches the image part; and
a dichroic mirror configured to selectively transmit or reflect the excitation light and the emission light.

4. The contact-type miniaturized ocular surface imaging system of claim 3, wherein the excitation light transfer part or the emission light transfer part comprises a depth-of-focus adjustment part configured to adjust a depth of focus.

5. The contact-type miniaturized ocular surface imaging system of claim 4, wherein the depth-of-focus adjustment part comprises an electronic tunable lens or a tube lens position adjustment part.

6. The contact-type miniaturized ocular surface imaging system of claim 3, wherein the excitation light transfer part or the emission light transfer part comprises a depth extension adjustment part configured to extend a depth of focus.

7. The contact-type miniaturized ocular surface imaging system of claim 2, wherein the positioning module comprises:
a 3D stage configured to move the imaging module in the X, Y, and Z directions; and
a rotation stage configured to rotate the imaging module in the Z direction.

8. The contact-type miniaturized ocular surface imaging system of claim 7, wherein the rotation stage comprises a rotation shaft and a rotation plate mounted on an end of the rotation shaft, and
a support member of the imaging module is disposed on an upper surface of the rotation plate at a position farther than the rotation shaft, along a straight line connecting the center of the ocular surface and the rotation shaft, and is configured to rotate according to a curvature of the ocular surface.

9. The contact-type miniaturized ocular surface imaging system of claim 1, wherein the image part comprises a camera of a smart device.

10. The contact-type miniaturized ocular surface imaging system of claim 9, wherein, when the image part is the camera of the smart device, the system further comprises, between the optical transfer part and the image part:
a smart device connection part configured to be connected to the camera of the smart device; or
a smart device focus adjustment part configured to adjust a focus of the smart device.

11. The contact-type miniaturized ocular surface imaging system of claim 1, wherein a wavelength of the excitation light is 405 nm, and
the emission light is a fluorescence signal caused by moxifloxacin.
